(19) United States
(12) Patent Application Publication
(10) Pub. No.: US 2010/0161004 A1
(43) Pub. Date: Jun. 24, 2010

(54) WIRELESS DYNAMIC POWER CONTROL OF AN IMPLANTABLE SENSING DEVICE AND METHODS THEREFOR

(75) Inventors: Nader Najafi, Ann Arbor, MI (US); Fred Brauchler, Canton, MI (US); Vincent Cruz, Farmington Hills, MI (US)

Correspondence Address: HARTMAN & HARTMAN, P.C., 552 EAST 700 NORTH, VALPARAISO, IN, 46383, US

(73) Assignee: INTEGRATED SENSING SYSTEMS, INC., Ypsilanti, MI (US)

(21) Appl. No.: 12/645,426

(22) Filed: Dec. 22, 2009

Related U.S. Application Data
(60) Provisional application No. 61/203,400, filed on Dec. 22, 2008.
(60) Provisional application No. 61/203,401, filed on Dec. 22, 2008.
(60) Provisional application No. 61/268,731, filed on Jun. 17, 2009.

(51) Int. Cl. A61N 1/08

(52) U.S. Cl. 607/60

(57) ABSTRACT

Communication systems and methods for dynamically controlling the power wirelessly delivered by a remote reader unit to separate sensing device, such as a device adapted to monitor a physiological parameter within a living body, including but not limited to intraocular pressure, intracranial pressure (ICP), and cardiovascular pressures that can be measured to assist in diagnosing and monitoring various diseases. The communication method entails electromagnetically delivering power from at least one telemetry antenna within the reader unit to at least one telemetry antenna within the sensing device, and controlling the power supplied to the sensing device within a predetermined operating power level range of the sensing device.

### CROSS REFERENCE TO RELATED APPLICATIONS

This application claims the benefit of U.S. Provisional Application Nos. 61/203,400 and 61/203,401, both filed Dec. 22, 2008, and U.S. Provisional Application No. 61/268,731 filed Jun. 17, 2009. The contents of these prior patent applications are incorporated herein by reference.

### BACKGROUND OF THE INVENTION

The present invention generally relates to implantable medical devices and to communication schemes and medical procedures performed therewith. More particularly, this invention relates to systems and methods for dynamically controlling power wirelessly delivered to such devices.

Wireless devices such as pressure sensors have been implanted and used to monitor various physiological parameters of humans and animals, including but not limited to heart, brain, bladder and ocular function. With this technology, capacitive pressure sensors are often used, by which changes in pressure cause a corresponding change in the capacitance of an implanted capacitor. The change in capacitance can be sensed, for example, by sensing a change in the resonant frequency of a tank or other circuit coupled to the implanted capacitor.

Telemetric implantable sensors that have been proposed include batteryless pressure sensors developed by CardioMEMS, Inc., Remon Medical, and the assignee of the present invention, Integrated Sensing Systems, Inc. (ISSYS). For example, see commonly-assigned U.S. Pat. Nos. 6,926,670 and 6,968,734 to Rich et al., and N. Najafi and A. Ludomirsky, “Initial Animal Studies of a Wireless, Batteryless, MEMS Implant for Cardiovascular Applications,” Biomedical Microdevices, 6:1, p. 61-65 (2004). With such technologies, pressure changes are typically sensed with an implant equipped with a mechanical (tuning) capacitor having a fixed electrode and a moving electrode, for example, on a diaphragm that deflects in response to pressure changes. The implant is further equipped with an inductor in the form of a fixed coil that serves as an antenna for the implant, such that the implant is able to receive a radio frequency (RF) signal transmitted from outside the patient to power the circuit, and also transmit the resonant frequency as an output of the circuit that can be sensed by an interrogator/reader unit outside the patient. Tele-powered implants of this type, as well as RFID (radio frequency identification) transponders, require an interrogator/reader unit equipped with an antenna to generate a sufficiently strong electromagnetic field capable of being received by the antenna of the implant. In the USA, the FCC (Federal Communications Commission) allows radio frequency devices to transmit in specific industrial, scientific, and medical (ISM) frequency bands ranging from 125 kHz to 2.4 GHz. The higher frequencies (greater than 100 MHz) suffer from tissue absorption and cannot easily be used for deeply implanted devices. Of the lower frequencies (less than 100 MHz), the 13.56 MHz ISM band is often used due to its compatibility with the desire to minimize the size of the coil and resonant capacitor of an implant.

For certain applications, the implant may be placed just below the skin or otherwise in proximity to an accessible external location, for example, within the eye to monitor intraocular pressure in the treatment of glaucoma disease. However, in order to monitor certain other parameters, including cardiovascular pressures to diagnose and monitor cardiovascular diseases such as chronic heart failure (CHF) and congenital heart disease (CHD) and intracranial pressure (ICP) to diagnose and monitor intracranial hypertension (ICH), the implant is typically placed farther from an accessible external location, for example, directly within a heart chamber whose pressure is to be monitored or in an intermediary structure, for example, the atrial or ventricular septum of the heart. Consequently, while communication distances of a few centimeters are sufficient for some applications, greater communication distances, for example, fifteen centimeters or more, would be desirable for others.

A complication of greater communication distances is that, for the lower communication frequencies (including the 13.56 MHz ISM band), the electromagnetic field generated by the reader appears nearly purely magnetic, and its level largely varies in inverse proportion to the distance between the reader and implant antennas. Consequently, the power coupled into an implant can vary by a factor of one hundred or more, depending on the location of the implant relative to the reader. In a typical RFID application, excess power supplied to an RFID device can be dissipated as heat since digital data typically read from RFID devices are typically not prone to erroneous measurements due to heat or temperature gradients. However, physiological parameters such as temperature and pressure can be distorted by excessive power delivered to a tele-powered implant. Accordingly, to promote the performance of a tele-powered implant device, power delivery and/or absorption should be compensated for or regulated in some manner. Implants equipped with a MEMS (microelectromechanical system) pressure transducer typically require a temperature sensor to provide for temperature compensation. Though systematic errors attributable to constant temperature gradients or peculiar transfer characteristics can be overcome by calibration, attempts to regulate and dissipate excess absorbed power within an implant will often result in localized heating and temperature gradients within the implant, including the temperature sensor, contributing to erroneous temperature measurements and, therefore, erroneous pressure measurements. As such, varying power dissipation levels within an implant can cause uncertainty due to the effects on the operation of the temperature sensor.

Excess power dissipation can also be detrimental to the transducer parameter extraction circuit used in implants. In the example of a MEMS pressure transducer, the extraction circuitry may be a capacitance-controlled relaxation oscillator (CCO) that transforms the MEMS capacitance into a frequency tone. Such circuitry depends on an on-chip ploy-resistor that has a temperature dependant resistance (for example, Tc=3500 ppm/° C.). Temperature uncertainty resulting from localized heating is reflected in the relaxation time and hence the oscillator frequency. Because the frequency tolerance of CCO relaxation oscillators demands a very low temperature variation or uncertainty (for example, less than 0.03° C.), even a small amount of excess power cannot be tolerated in the implant, necessitating some type of management scheme.

### BRIEF SUMMARY OF THE INVENTION

The present invention provides communication systems and methods for dynamically controlling the power wirelessly delivered by a remote reader unit to a separate sensing device, such as a device adapted to monitor a physiological parameter within a living body, including but not limited to intraocular pressure, intracranial pressure (ICP), and cardiovascular pressures that can be measured to assist in diagnosing and monitoring various diseases. According to a particular aspect of the invention, such a communication system can be adapted to provide enhanced functionality and data rate transfers by combining digital and analog communication between the sensing device and reader unit.

The communication system includes at least one telemetry antenna within the reader unit and adapted for electromagnetically delivering power to the sensing device, at least one sensing element within the sensing device for sensing a parameter of the fluid and producing an output based on the parameter, electronic components within the sensing device for processing the output of the sensing element and generating therefrom a processed data signal of the sensing device, and at least one telemetry antenna within the sensing device for receiving the power electromagnetically delivered by the reader unit and communicating the processed data signal to the reader unit. The electronic components are adapted to be powered at an operating power level. The communication further includes means for preventing the power supplied to the electronic components from exceeding the operating power level.

The communication method generally entails a reader unit and sensing device that can be of the type described above, and involves electromagnetically delivering power from a telemetry antenna within the reader unit to a telemetry antenna within the sensing device, and preventing the power supplied to electronic components of the sensing device from exceeding the operating power level.

The communication scheme and method are particularly intended for use with wireless implantable medical devices that obtain all of their power from a reader unit located outside the body, enabling safe, detailed, real-time, and continuous monitoring of a physiological parameter. According to a preferred aspect of the invention, excess power supplied to the device can be avoided, thereby eliminating the requirement to dissipate heat, avoiding potential measurement errors arising from localized heating or temperature gradients within the device, and avoiding unnecessary heating of tissue that surrounds the device when implanted in a body.

Other aspects and advantages of this invention will be better appreciated from the following detailed description.

### BRIEF DESCRIPTION OF THE DRAWINGS

FIGS. 1 and 2 schematically represent implantable devices of types that can be employed in the present invention.

FIG. 3 is a block diagram of a wireless pressure monitoring system utilizing a passive sensing scheme that can be utilized by the present invention.

FIGS. 4 through 6 schematically represent communication schemes for dynamically controlling power that is wirelessly delivered to an implantable device, for example, of the types depicted in FIGS. 1 and 2, in accordance with three embodiments of this invention.

FIG. 7 is a graph representing an encoding scheme that can be used with the invention to transmit sampled data from an implantable device to a remote reader unit.

FIG. 8 is a block diagram representing a communication protocol that can be used with the invention to transmit information between an implantable device and a remote reader unit.

FIG. 9 is a graph representing a reader-to-sensor protocol that can be used with the invention to transmit information from an implantable sensing device to a remote reader unit.

### DETAILED DESCRIPTION OF THE INVENTION

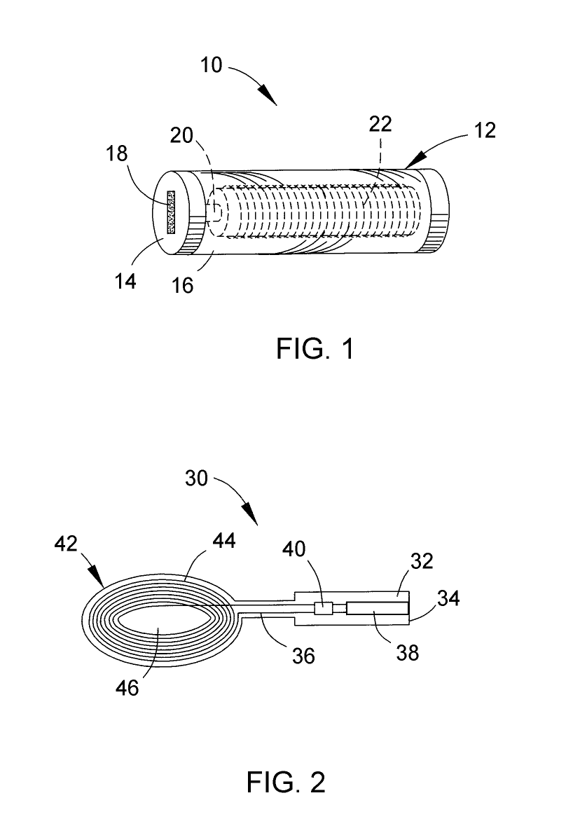

FIG. 1 schematically depicts one example of an implantable sensing device 10 of a type that can be used with the present invention. The device 10 is represented as having a cylindrical housing 12, which is convenient for placing the sensing device 10 within certain types of anchors adapted to secure the sensing device 10 to or within a wall-like structure, for example, the skull or the atrial or ventricular septum of the heart. Other exterior shapes for the housing 12 are also possible to the extent that the exterior shape permits placement of the sensing device 10 in a desired location or assembly of the sensing device 10 with an anchor. The cylindrical-shaped housing 12 of FIG. 1 includes a flat distal face 14, though other shapes are also possible, for example, a torpedo-shape in which the peripheral face 16 of the housing 12 immediately adjacent the distal face 14 is tapered or conical (not shown). The housing 12 can be formed of glass, for example, a borosilicate glass such as Pyrex Glass Brand No 7740 or another suitable material capable of forming a hermetically-sealed enclosure for the electrical components of the sensing device 10. A biocompatible coating, such as a layer of a hydrogel, titanium, nitride, oxide, carbide, silicide, silicone, parylene and/or other polymers, can be deposited on the housing 12 to provide a non-thrombogenic exterior for the biologic environment in which the sensing device 10 will be placed. A nonlimiting example of an overall size for the housing 12 is about 3.7 mm in diameter and about 16.5 mm in length.

As schematically depicted in FIG. 1, the sensing device 10 includes a transducer 18 located at the flat distal face 14, and the housing 12 contains electronics 20 and an antenna 22, the latter of which occupies most of the internal volume of the housing 12. The transducer 18 can be adapted to sense a variety of parameters, including but not limited to pressure. The transducer 18 is preferably a MEMS device, more particularly a micromachine fabricated by additive and subtractive processes performed on a substrate. The substrate can be rigid, flexible, or a combination of rigid and flexible materials. Notable examples of rigid substrate materials include glass, semiconductors, silicon, ceramics, carbides, metals, hard polymers, and TEFLON. Notable flexible substrate materials include various polymers such as parylene and silicone, or other biocompatible flexible materials. A particular but nonlimiting example of the transducer 18 is a MEMS capacitive pressure sensor for sensing pressure, such as bariatric pressure, blood pressure, or intracranial pressure (ICP) of cerebrospinal fluid. A nonlimiting example of a preferred MEMS capacitor has a gauge pressure range of about −100 to about +300 mmHg, an absolute pressure range of about 300 mmHg to 1500 mmHg, and an accuracy of about 1 mmHg. A variety of additional or other sensing elements could be incorporated into the sensing device 10, for example, inductive, resistive, and piezoelectric sensing elements could be used. Furthermore, the transducer 18 could be configured to sense temperature, flow, acceleration, vibration, pH, conductivity, dielectric constant, and chemical composition, including the composition and/or contents of a sensed fluid. Though the transducer 18 is shown located on the flat distal face 14 of the cylindrical housing 12, the transducer 18 can be located at various locations near the distal end of the sensing device 10, for example, on the peripheral face 16 of the housing 12 immediately adjacent the distal face 14. The distal face 14 can be defined by a biocompatible semiconductor material, such as a heavily boron-doped single-crystalline silicon, in whose outer surface the transducer 18 (for example, a pressure-sensitive diaphragm of a capacitor) is formed. In this manner, only the distal face 14 of the housing 12 need be in contact with the media being sensed, such as blood, cerebrospinal fluid, etc., whose physiological parameter is to be monitored.

The size and location of the antenna 22 are governed by the need to couple to a magnetic field to enable tele-powering of the sensing device 10 when implanted within the body using a remote interrogator/reader unit located outside the body, as will be discussed in more detail below. The antenna 22 generally comprises a coil assembly that can be made using any method known in the art, such as winding a conductor around a ferrite core, depositing (electroplating, sputtering, evaporating, screen printing, etc.) a conductive coil (preferably made from a highly conductive metal such as silver, copper, gold, etc.) on a rigid or flexible substrate), or any other method known to those skilled in the art. As such, the antenna 22 can be flat or three-dimensional such as cylindrical (as represented in FIG. 1), cubic, etc.

An advantage of a flat configuration is that it can be easily implanted under the skin, such as between the scalp and skull so that the antenna 22 lies flat against the skull. Such an embodiment is represented in FIG. 2, which represents an implantable sensing device 30 configured to have a housing 32 that contains a transducer 38 located adjacent a distal end 34 of the housing 32 and electronics 40, and is coupled to an external flexible antenna 42. This type of device 30 is adapted for deep implantation of the housing 32 within the body, for example, the brain, while permitting the antenna 22 to be located remote from the device 30. The antenna 42 can be fabricated by forming a coil 44 on a flexible or rigid film 46, which can be formed of any suitable biocompatible material. The antenna 42 is shown as physically and electrically interconnected with the housing 32 by a cable 36, which may be flexible, rigid, or combination of flexible and rigid. The cable 36 may be coated, potted or covered with a biocompatible material.

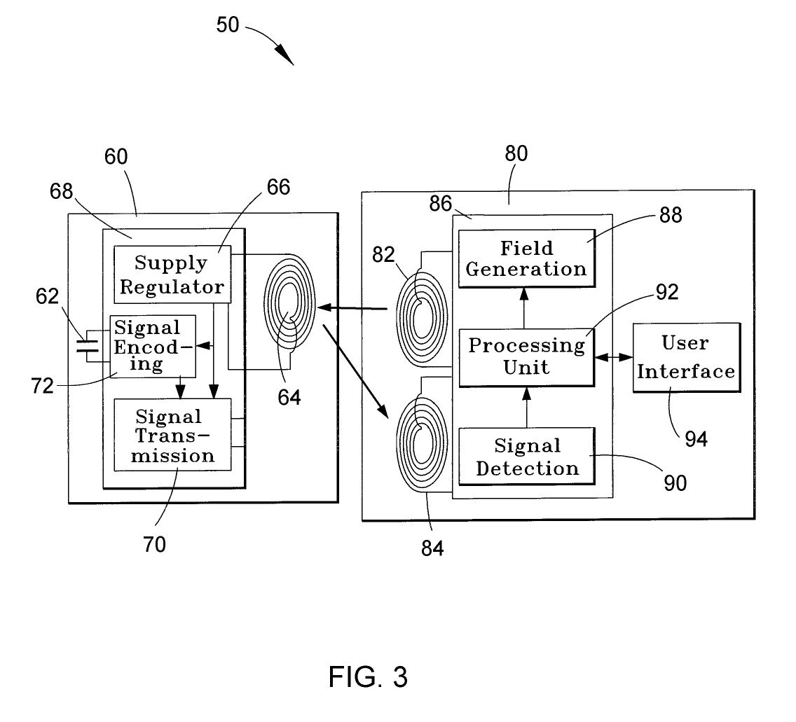

FIG. 3 schematically illustrates a monitoring system 50 and components thereof capable of implementing the implantable sensing devices 10 and 30 of FIGS. 1 and 2, as well as various other implantable sensing devices within the scope of the invention. An implantable sensing device and its companion interrogator/reader unit (hereinafter, reader unit) are identified by reference numbers 60 and 80 in FIG. 3. The reader unit 80 is adapted to wirelessly communicate with the sensing device 60 while the sensing device 60 is implanted at a desired location within a body. Because the sensing device 60 and reader unit 80 wirelessly communicate with each other, the monitoring system 50 lacks a wire, cable, tether, or other physical component that conducts the output of the sensing device 60 to the reader unit 80. As such, the sensing device 60 defines the only implanted portion of the monitoring system 50.

FIG. 3 represents the sensing device 60 and reader unit 80 as configured to perform a wireless pressure sensing scheme disclosed in U.S. Pat. Nos. 6,926,670 and 6,968,734 to Rich et al. A wireless telemetry link is established between the sensing device 60 and reader unit 80 using a passive, magnetically-coupled scheme, in which onboard circuitry of the sensing device 60 receives power from the reader unit 80. FIG. 3 depicts the sensing device 60 as containing a transducer 62 and an antenna 64 represented as an inductor coil. The transducer 62 is represented in FIG. 3 as being in the form of a pressure sensor, and more specifically a mechanical capacitor adapted to sense pressure as a physiological parameter of interest. In addition to sensing physiological parameters, the sensing device 60 can be configured to include various actuation functions, including but not limited to thermal generators, voltage and/or current sources, probes, and/or electrodes, drug delivery pumps, valves, and/or meters, microtools for localized surgical procedures; radiation-emitting sources, defibrillators, muscle stimulators, pacing stimulators, etc.

As a passive communication scheme, the sensing device 60 lacks any internal means to power itself lies and therefore lies passive in the absence of the reader unit 80. When a pressure reading is desired, the reader unit 80 is brought within range of the antenna 64 of the sensing device 60 to enable magnetic coupling between the antenna 64 and a second antenna 82 associated with the reader unit 80. The antenna 82 is adapted to transmit an alternating electromagnetic field to the antenna 64 of the sensing device 60 and induce a sinusoidal voltage across the coil of the antenna 64. When sufficient voltage has been induced, a supply regulator 66 within the sensing device 60 converts the alternating voltage on the antenna 64 into a direct voltage that can be used by electronics 68 as a power supply for signal conversion and communication. At this point the sensing device 60 can be considered alert and ready for commands from the reader unit 80. To minimize the size of the sensing device 60, the antenna 64 may be employed for both reception and transmission, or the sensing device 60 may utilize the antenna 64 solely for receiving power from the reader unit 80 and employ a second antenna (not shown) for transmitting signals to the reader unit 80.

The supply regulator 66 contains rectification circuitry that preferably outputs a constant voltage level for the other electronics from the alternating voltage input from the antenna 64. The rectification circuitry can be of any suitable type, including but not limited to full-bridge diode rectifiers, half-bridge diode rectifiers, and synchronous rectifiers. The rectification circuitry may further include a capacitor for transient energy storage to reduce the noise ripple on the output supply voltage. The supply regulator 66 is represented as implemented on the same integrated circuit die as other components of the sensing device electronics 68, for example, an application-specific integrated circuit, or ASIC. As represented in FIG. 3, the device electronics 68 include signal transmission circuitry 70 that receives an encoded signal generated by signal conditioning circuitry 72 based on the output of the transducer 62, and then generates a signal that is propagated to the reader unit 80 with the antenna 64.

A benefit of configuring the sensing device 60 without a battery is that the device 60 and its operation do not require replacement or charging of a battery, and the size of the device 60 is not dictated by the need to accommodate a battery. However, the sensing device 60 of FIG. 3 could be modified to use one or more batteries or other power storage devices to power the sensing device 60 when the reader unit 80 is not sufficiently close to induce a voltage in the sensing device 60. Furthermore, it is also within the scope of the invention that such power storage devices may be rechargeable and capable of being recharged with the reader unit 80.

In addition to the antenna 82 for communicating with and powering the sensing device 60, the reader unit 80 is represented in FIG. 3 as including a separate antenna 84 for receiving the signals transmitted by the antenna 64 of the sensing device 60, and front-end electronics 86 for processing the signal of the sensing device 60 as well as generating the alternating electromagnetic field sent by the antenna 82 to the sensing device 60. For purposes of compactness, the functions of the antennas 82 and 84 could be performed by a single antenna. The front-end electronics 86 include field generation circuitry 88 for generating the alternating electromagnetic field generated by the antenna 82, signal detection circuitry 90 for receiving data transmitted by the antenna 64 of the sensing device 60, and a processing unit 92 that processes the data received through the detection circuitry 90, relays the processed data to a user interface 94, and enables control of the field generation circuitry 88. The fabrication and operation of the front-end electronics 88 and its components are well known in the art and therefore will not be discussed in any detail here. The user interface 94 may be a display, computer, or other data logging devices that can be physically incorporated into the reader unit 80 or separate and coupled to the unit 80 through a cable or wirelessly.

As alternatives to the sensing scheme of FIG. 3, wireless telemetry links can be established using other schemes, such as a resonant scheme also disclosed in U.S. Pat. Nos. 6,926,670 and 6,968,734 to Rich et al. or a fully or partially active scheme in which the sensing device 60 may contain batteries and/or rechargeable power storage devices. In a resonant scheme, the sensing device contains a packaged inductor coil (similar to the antenna 64 of FIG. 3) and a pressure sensor in the form of a mechanical capacitor (similar to the capacitor 62 of FIG. 3), which together form an LC (inductor-capacitor) tank resonator circuit that has a specific resonant frequency, expressed as 1/(LC)^{1/2}, that can be detected from the impedance of the circuit. At the resonant frequency, the circuit presents a measurable change in magnetically-coupled impedance load to an external antenna associated with a separate reader unit (similar to the antenna 82 and reader unit 80 of FIG. 3). Because the resonant frequency is a function of the capacitance of the capacitor within the sensing device, the resonant frequency of the LC circuit changes in response to pressure changes that alter the capacitance of the capacitor. Because the coil within the sensing device has a fixed inductance value, the reader unit is able to determine the pressure sensed by the sensing device by monitoring the resonant frequency of the circuit.

A wireless communication platform implemented with the monitoring system 50 should take into consideration a number of important aspects. Regarding data sample bandwidth, the sampling rate should be greater than 200 Hz for some applications to achieve high resolution and clinically useful data when monitoring many biologic parameters, such as cardiovascular and intracranial pressures. As an example, AAMI standards for blood pressure monitoring specify a 200 Hz cutoff frequency. The sensing devices (e.g., 10, 30 and 60 in FIGS. 1, 2 and 3) and their reader units (e.g., 80 in FIG. 3) should also be capable of communicating distances as required for communication between internal organs intended to be monitored and the nearest accessible locations outside of the body. As previously noted, while a few centimeters of communication can be sufficient for some applications, a communication distance of fifteen centimeters or more will be desirable or necessary for others. Finally, the sensing devices 10, 30 and 60 should ideally be capable of being delivered to the site of implantation with a catheter not larger than French 15 size (about 5 mm in diameter), and preferably French 11 (about 3.7 mm in diameter), which establishes limitations on the type and size of electronics within the housing (e.g., 12 and 32) of the sensing device 10, 30 and 60. On the other hand, greater coil size corresponds to longer communication distances. Therefore, for the sensing device 10 of FIG. 1 (and other designs with an enclosed antenna), the antenna 22 should be as large as possible, necessitating that the electronics within the housing 12 be as small as possible to meet a desired package size. As an example, the coil of the antenna may have a maximum size of a few millimeters in diameter and a length of about ten to fifteen millimeters, and an ASIC die carrying the electronics may have a maximum width and length of about 2 mm. A wireless sensing device meeting these dimensional goals should be capable of delivery using minimally invasive procedures, have minimal impact on the body in which it is implanted, and be more readily accepted for research and clinical use.

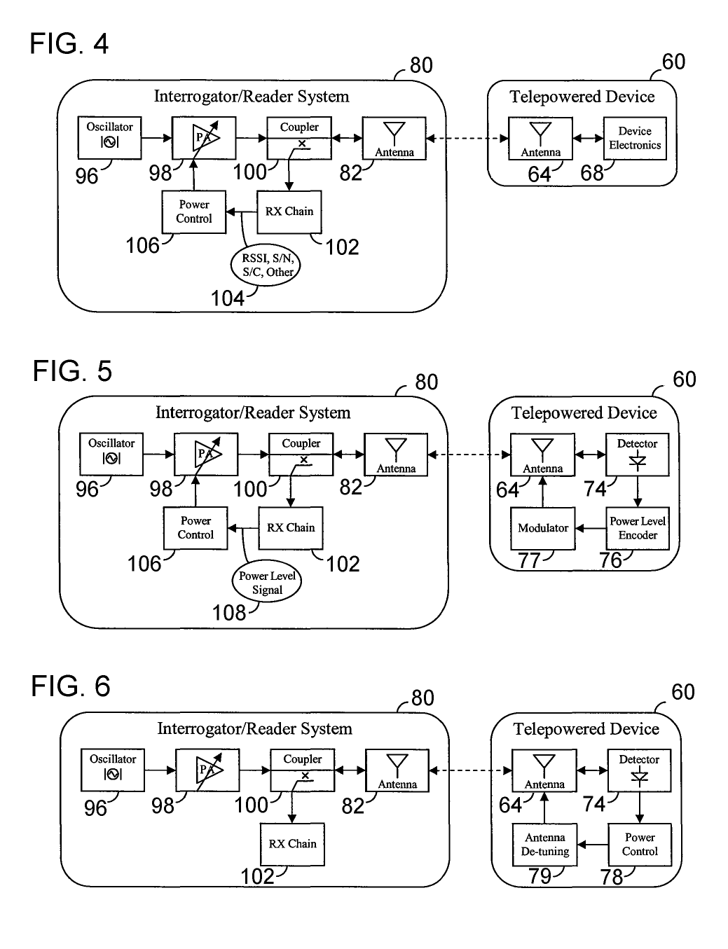

FIGS. 4 through 6 represent further aspects of the monitoring system 50 of FIG. 3 for achieving dynamic control of power delivered to the sensing device 60. Dynamic power control is provided for the purpose of compensating for potentially very large variations in the power level delivered to the sensing device 60 as a result of the likelihood that the transmission distance between the antennas 64 and 82 of the sensing device 60 and reader unit 80 will vary widely, depending on the location and use of the sensing device 60. The maximum achievable transmission distance between the antennas 64 and 82 (and, if present, the separate reception antenna 84) will be limited by various factors, including the magnetic field strength generated by the reader unit 80 and the quality and size of the antenna coil of the sensing device 60. As the transmission distance is reduced, more power is transmitted to the sensing device 60 and, if excessive, can lead to damage to the device 60, damage to body tissue surrounding the device 60, and sensor output errors. In the embodiments of FIGS. 4 through 6, power delivery is dynamically controlled to avoid the delivery of excess power to the sensing device 60, instead of relying on power dissipation within the device 60. As such, damage to the sensing device 60 and surrounding body tissue is avoided, as well as errors that can occur in the output of the sensing device 60 and its transducer 62 as a result of power oversupply and heating of the device 60. As a result, the embodiments of the monitoring system 50 represented in FIGS. 4 through 6 are capable of improving the accuracy and stability of the signal generated by the sensing device 60, and thereby provides a more accurate indication of the physiological parameter being monitored.

FIGS. 4 through 6 generally represent communication schemes that incorporate dynamic power control in accordance with three embodiments of the present invention. In FIG. 4, the reader unit 80 is adapted to control the power level delivered to the sensing device 60 using one or more feedback signals that are transmitted by the sensing device 60 and then received and processed by the reader unit 80. Such feedback signals may be based on signal strength, signal-to-noise ratio, signal-to-carrier ratio, etc., of the data transmission signal generated by the sensing device 60. In FIG. 5, power level control is accomplished using an interactive signal between the reader unit 80 and the sensing device 60. Finally, power level control is accomplished in FIG. 6 by varying the tank load resistance and/or reactance of the coil of the antenna 64 of the sensing device 60. For convenience, FIGS. 4 through 6 depict only those components of the sensing device 60 and the reader unit 80 that are particularly relevant to the description of the dynamic power control scheme, while others (including components represented in FIG. 3) are omitted. Furthermore, reference numbers used in FIG. 3 are also used in FIGS. 4 through 6 to identify the same or functionally equivalent components, and reference numbers used in FIGS. 4 through 6 to identify additional components are consistently used throughout FIGS. 4 through 6 to identify the same or functionally equivalent components employed in the embodiments.

With reference to FIG. 4, powering of the sensing device 60 does not contain any means for providing direct feedback/communication from the sensing unit 60 to the reader system 80, and there are no direct means of assessing the power level delivered by the reader unit 80 to the sensing device 60 or providing feedback of the power level to the reader unit 80 to the sensing device 60. Instead, the sensing device 60 relies entirely on the reader unit 80 to determine the appropriate power level delivered to the sensing device 60. The reader unit 80 contains components for evaluating an internal receiver signal characteristic of the sensing device 60, including but not limited to receive signal strength indicator (RSSI), signal-to-noise ratio (S/N), signal-to-carrier ratio (S/C), minimum (or desired) detectable signal strength, etc., to determine what power level should be delivered to the device 60. FIG. 4. depicts the sensing device 60 as containing the antenna 64 and electronics 68, corresponding to the components represented in FIG. 3. Similarly, the reader unit 80 is shown in FIG. 4 as containing the antenna 82 corresponding to the antenna 82 represented in FIG. 3 and, as such, the antenna 82 creates a magnetic (electromagnetic) field that powers the antenna 64 of the sensing device 60. (In FIGS. 4 through 6, the second antenna 84 is omitted and its reception function merged into the antenna 82.) The reader unit 80 further includes an oscillator 96 which sets the carrier frequency and drives a power amplifier (PA) 98. According to a preferred aspect of this embodiment, the power amplifier 98 has a variable gain and hence a variable output signal amplitude. The amplified signal drives the antenna 82 through a directional coupler 100. Signals returning from the sensing device 60 via the antenna 82 are sampled by the directional coupler 100 and processed by a receiver (RX) chain 102. In this embodiment, one or more signal parameters 104 characteristic of the communication link between the sensing device 60 and reader unit 80 are examined to assess and control the output signal amplitude (power level) transmitted by the antenna 82. A power control 106 uses the signal parameters 104 to assess the power level being received by the sensing device 60 and then, if necessary, adjusts the output signal amplitude of the power amplifier 98 to a level that will avoid overpowering the sensing device 60.

Nonlimiting examples of signal parameters 104 of particular interest are represented in FIG. 4 as including RSSI, S/N, S/C and combinations thereof, which can be used individually or in combination to provide an indication as to the proximity of the sensing device 60 to the reader unit 80 or the distance between the antennas 64 and 82 of the device 60 and reader unit 80 based on information sent by the sensing device 60 to the reader unit 80. For example, RSSI can be used by the reader unit 80 to estimate the strength, quality or amount of power received by the sensing device 60, and therefore an indication of the distance between the sensing device 60 to the reader unit 80, which is then used by the reader unit 80 to enable the power control 106 to adjust the output signal amplitude of the power amplifier 98 as needed.

In contrast to the embodiment of FIG. 4, FIG. 5 represents an embodiment that relies on a feedback signal from the sensing device 60 to adjust the power level transmitted by the reader unit 80 to the device 60. In this case, the sensing device 60 requires power level detection, modulator control, and antenna modulation circuitry to sense and transmit information regarding the power level back to the reader unit 80, which then determines whether the power level being received by the sensing device 60 is adequate (within a predetermined range) or above or below a predetermined threshold, and if necessary adjusts the power level transmitted to the sensing device 60 until a targeted power level is achieved.

Similar to FIG. 4, the reader unit 80 is represented in FIG. 5 as comprising an antenna 82, oscillator 96, power amplifier (PA) 98, directional coupler 100, receiver (RX) chain 102, and power control 106. Unless otherwise indicated, these components perform the same operations as described for FIG. 4. In contrast to FIG. 4, the sensing device 60 contains a power detector 74 adapted to assess the power level received by the antenna 64 of the sensing device 60, and then provide such information to a power level encoder 76. The power level encoder 76 dictates information that is encoded by a modulator 77 onto the antenna 64. In particular, in addition to the signal pertaining to the measurements performed by the sensing device 60, the power level encoder 76 drives the modulator 77 to encode information pertaining to the power level received by the sensing device 60, and specifically whether the power level is within or outside a predetermined range for the sensing device 60. When this information is received by the reader unit 80, the information is sampled by the directional coupler 100 and processed by the RX chain 102. In this embodiment, the power level signal 108 is extracted by the RX chain 102 and directly used by the power control 106 to adjust, if necessary, the output signal amplitude of the power amplifier 98 to ensure that the sensing device 60 is continuously receiving an appropriate power level.

Alternatively, in FIG. 5. the sensing device 60 may be equipped to produce a signal that offers a much wider spectrum, for example, analog or higher numbers of digital values. The specific indicator signal may be digital or analog or a combination thereof. In one embodiment, if the power level is too low or is decreasing beyond a certain level the sensing device 60 can be configured to drop its transmission frequency to a another value (for example, 30% below the normal operating frequency or to a specific pre-determined frequency outside the normal operation range), and if the power level is too high or is increasing above a certain level the sensing device 60 may push its transmission frequency to a another value (for example, 30% above the normal operating frequency or to a specific pre-determined frequency outside the normal operation range). Finally, the sensing device 60 may be configured to simply control an indicator on the reader unit 80 that allows the operator to manually select the power level generated by the reader unit 80. In addition, either the sensing device 60 or reader unit 80, or both may incorporate other means for indicating the proximity of the sensing device 60 to the reader unit 80, such as a proximity sensor, for example, a capacitive or ultrasonic sensor that determines the distance between the reader unit 80 and the sensing device 60. The sensing device 60 may include various other components capable of generating a specific indicator signal to indicate whether the power received by the sensing device 60 is within an acceptable range. Such a component may generate a signal indicating low power and another for excess power.

The third embodiment of FIG. 6 simplifies the reader unit 80 by transferring the entire dynamic power control function to the sensing device 60. In this case, the power level is detected and fed into a power control circuit within the sensing device 60, which itself controls the power level that can be coupled into the device 60 by the antenna 64. In a preferred aspect of this embodiment, the power level transmitted by the reader unit 80 is detected and controlled via antenna tank load de-tuning within the sensing device 60. Similar to FIGS. 4 and 5, the reader unit 80 is represented in FIG. 6 as comprising an antenna 82, oscillator 96, power amplifier (PA) 98, directional coupler 100, receiver (RX) chain 102, and power control 106. Unless otherwise indicated, these components perform the same operations as described for FIGS. 4 and 5.

As with the prior embodiments, the oscillator 96 sets the carrier frequency and drives the power amplifier 98, the output signal of the power amplifier 98 drives the antenna 82 through the directional coupler 100, and the antenna 82 generates a magnetic (electromagnetic) field for powering the sensing device 60. In contrast to the prior embodiments, the power amplifier 98 can have a fixed gain and hence a fixed output signal amplitude level. The antenna 64 of the sensing device 60 couples to the magnetic field generated by the reader unit 80 for powering the sensing device 60. As in the embodiment of FIG. 5, the sensing device 60 includes a power detector 74 for assessing the power level transmitted by the reader unit 80 and received by the antenna 64, and provides that information to a power control 78 that dictates the state that an antenna de-tuner 79 applies to the antenna 64. The de-tuner 79 controls the tank mismatch or load circuit of the antenna 64. If the power level received by the antenna 64 is within a predetermined range for the sensing device 60, the power control 74 drives the antenna de-tuner 79 to maintain the operation of the antenna 64. If the power level is above or below the predetermined range, the power control 78 drives the antenna de-tuner 79 to increase or decrease, respectively, the tank load resistance and/or reactance, thereby adjusting the power absorbed by the antenna 64. If the power level transmitted by the reader unit 80 is above a predetermined threshold, the antenna mismatch load is increased to reject the extra power transmitted by the reader unit 80. Conversely, if the internal power level of the sensing device 60 is below a predetermined threshold, the antenna mismatch load is reduced to increase the power coupled into the device 60 by the antenna 64.

In contrast to the embodiments of FIGS. 4 and 5, no information related to the power level at the sensing device 60 needs to be communicated back to the reader unit 80 in the embodiment of FIG. 6. Nonetheless, features of the first and second embodiments can be incorporated into the embodiment of FIG. 6 to provide coarse power setting or provide further indicators of power level for reasons other than power control, such as signal indication. For example, at the extremes of the power control range, the embodiment of FIG. 6 can be modified to provide a feedback signal that may be used as described for the embodiment of FIG. 5, or can simply be used as a range indicator.

It is foreseeable that a combination or combinations of the three embodiments described above could be used, in which both the sensing device 60 and the reader unit 80 manage the dynamic power control. In such embodiments, the output of the power amplifier 98 is controlled as well as antenna de-tuning performed by the de-tuner 79 of sensing unit 60.

In view of the above, each of the embodiments of FIGS. 4, 5 and 6 provides a power control technique in the sensing device 60 to mitigate excess powering of the device 60. As such, the invention can prevent damage to the device 60, prevent heating and damage to surrounding body tissue, enable more accurate and stable sensing information, as well as other benefits as a result of avoiding incidences of the sensing device 60 receiving excessive power from the reader unit 80. In medical-related implants, a more significant effect is the avoidance or at least a significant reduction in measurement errors resulting from excessive power supplied to the components of the sensing device 60 and/or localized heating of the components attributable to receiving excessive power levels. For example, the invention avoids or at least mitigates sensing errors that can occur as a result of excessive powering and/or localized heating of a temperature sensor used to compensate the output of the transducer 62 for variations in temperature, and/or avoids or at least mitigates output errors that can occur in the output of the transducer 62 itself as a result of the transducer 62 receiving excess power and/or localized heating of the transducer 62 attributable to receiving excess power.

The embodiments of the invention described above, as well as a variety of other monitoring systems, can be modified to make use of a wireless communication platform that transmits both digital and analog data. As will become apparent from the following description, the mixed analog and digital communication is capable of both enhanced functionality via digital communication while allowing higher sensor data rates (or other information) via analog communication. Furthermore, the analog communication can eliminate the need for an analog-to-digital convertor in a sensing device (such as one of the sensing devices 60 described above), which is advantageous since such converters can consume considerable power and may add noise to the signal transmitted by the sensing device. Additional potential advantages include the ability to reduce the size of the sensing device and increase transmission distances and the potential for longer sensor life when monitoring physiological parameters of the human body. In addition, the wireless communication platform can enable bi-directional communication that could allow for actively responding to individual needs, such as closed-loop drug delivery.

The wireless communication platform is particularly well suited for the magnetic telemetry technique described above for the sensing device 60 and reader unit 80, though other technologies (including but not limited to ultrasonic telemetry techniques) could be employed. In a preferred application of this platform, a passive communication scheme as described above for the reader unit 80 and the sensing device 60 is employed, meaning that the sensing device 60 does not contain a battery and receives all of its operating power from the reader unit 80, though an active scheme utilizing a power storage device (e.g., a battery) could also be used. In addition, the communication platform makes advantageous use of the second antenna 84 shown for the reader unit 80 of FIG. 3. Accordingly, the communication platform will be described in reference to the monitoring systems 50, sensing devices 10, 30 and 60, and reader unit 80 of FIGS. 1 through 6, though it should be understood that the communication platform is not limited to the particular embodiments disclosed and described for these figures.

Magnetic telemetry schemes of the type previously described for the sensing devices 10, 30 and 60 and reader unit 80 of FIGS. 1 through 6 have been proven and used extensively in the identification and tracking industry, for example, RFID tags. However, a number of modifications are desirable in order to implement the strictly digital identification technology employed by RFID tags to sensing applications suitable for medical implants. RFID technologies to do not employ an analog interface, and their protocols are not intended for sensors and other implants (such as actuators). Furthermore, traditional RFID magnetic telemetry schemes employ a single coil on the RFID tag to both receive power from a reader unit and also transmit information back to the reader unit. While convenient from a packaging perspective and minimizing costs, this approach may compromise the effectiveness of both the receiver and the transmitter coils in some applications. With this in mind, the following will describe a wireless communication platform that divides the functions of transmitting and receiving performed by the reader unit 80 between two separate coils, such as the antennas 82 and 84 in FIG. 3. In this way, the transmitting coil (82) can be optimized for communication with the sensing device 60, while simultaneously optimizing the receiving coil (84) for efficient capture of digital and analog signals from the sensing device 60. However, as with the embodiments of FIGS. 4 through 6, the transmission and reception functions could be merged onto a single antenna (e.g., 82 in FIGS. 4 to 6).

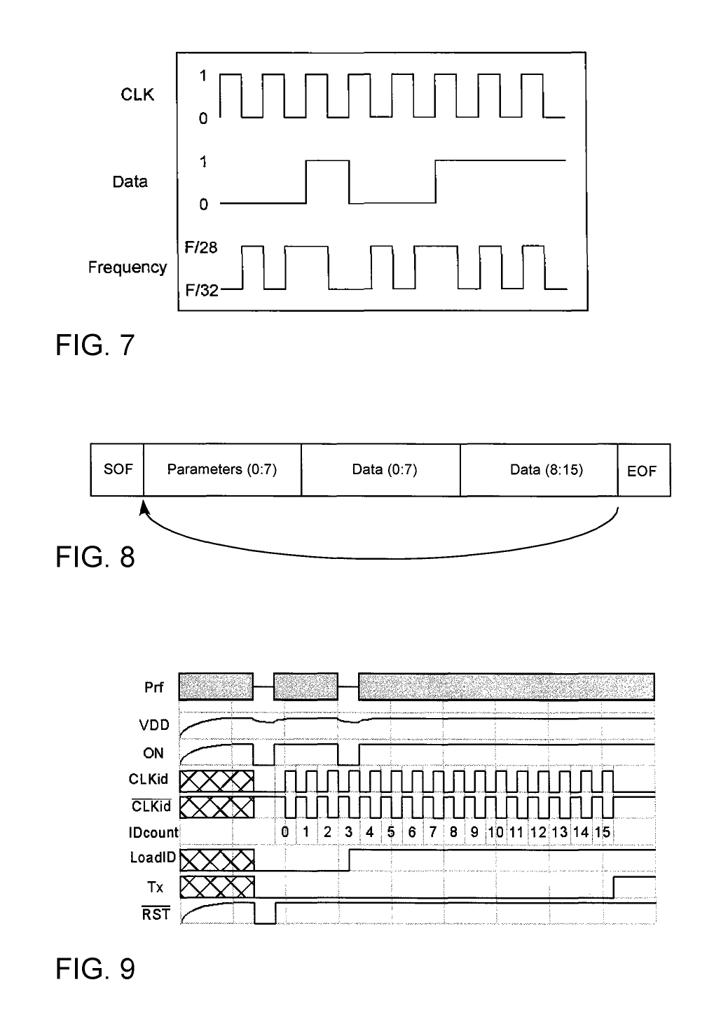

Modulation of sampled data onto the subharmonic carrier for transmission from the sensing device 10, 30 or 60 to the reader unit 80 can be accomplished with many schemes including analog modulation such as amplitude modulation (AM) frequency modulation (FM), and digital modulation such as phase shift keying (PSK) and frequency shift keying (FSK). For example, FSK modulation can be used to map two distinct frequencies to the digital bits 1 and 0. This particular coding scheme is very robust to interference, has adequate bandwidth, and is technologically mature. The FSK signal is then Manchester encoded to ensure proper timing synchronization between the sensing device 10, 30 or 60 and reader unit 80. FIG. 7 is illustrative of a suitable Manchester encoding scheme, which represents a bit transition from 0 to 1 or vice versa as occurring during the middle of the bit interval. This modulation/coding scheme is believed to offer a high level of immunity to noise and other interferences.

Because higher radio frequencies (above 100 MHz) suffer from tissue absorption, lower frequencies are preferred by the invention for the sensing devices 10, 30 and 60 when deeply implanted into the human body, such as within the heart. Of the lower frequencies, the 13.56 MHz ISM band is most attractive as the power transmission frequency from the reader unit 80 to the sensing device 10, 30 or 60 due to the minimal size required for the coil of the sensing device 10, 30 or 60 and its associated resonant capacitor. Both power transmission frequency from the reader unit 80 and the data transmission frequency from the sensing device 10, 30 or 60 should be optimized for optimum performance of the monitoring system 50. To select the FSK carriers and modulation rates, one will evaluate bandwidth capacity and noise immunity of all subharmonic bands of 13.56 MHZ down to 423.8 kHz. Tradeoffs for different frequencies may include signal-to-noise immunity, circuit size, power consumption, and transmitter antenna efficiency. The rate of FSK modulation should also be chosen in view of the direct tradeoff between bandwidth and noise immunity. The data transmission frequency from the sensing device 10, 30 and 60 to the reader unit 80 can be the same frequency or different from the power transmission frequency. A preferred subharmonic for FSK modulation of the data transmission frequency is believed to be 3.39 MHz, for reasons including a sufficiently high frequency to maintain transmission efficiency and transmit the required bandwidth, and sufficiently far enough from 13.56 MHz to allow for bandstop filters. In addition, this data transmission frequency allows for the use of a single coil for both reception and transmission of RF signals (digital and analog) with the sensing device 10, 30 or 60, thereby minimizing the required internal volume of the sensing device 10, 30 or 60.

In view of the above, a preferred modulation scheme between the reader unit 80 and the sensing device 10, 30 or 60 is believed to be digital transmission using a 13.56 MHz carrier frequency. For simultaneous transmission of both analog and digital information between the sensing device 10, 30 or 60 and the reader unit 80, a preferred modulation scheme is believed to include the following: 20-200 kHz modulation bandwidth, digital transmission using FSK modulation of an AM frequency (for example, Logic 0: AM frequency equal to 75.625 kHz, and Logic 1: AM frequency equal to 105.94 kHz), and analog transmission using frequency modulation (FM) of an AM frequency (for example, the analog signal is proportional to the AM frequency). In view of the foregoing, specific electronics for achieving these modulation schemes will be evident to those skilled in the art, and therefore will not be described in any detail here.

The protocol for communication between the sensing device 10, 30 or 60 and the reader unit 80 specifies an agreed order and content for transmitting information, and is an important aspect of a wireless communication platform used in the monitoring system 50 because it determines the complexity of electronics needed in the instrument. Particularly suitable protocols should allow simple electronics to perform basic operations while allowing for expanded capabilities, including communication between the reader unit 80 and a number of different sensing devices 10, 30 or 60 adapted to sense a variety of physiological parameters, in which case the protocol should also include a code that identifies the individual sensing devices, for example, by family and serial number. The protocol should also preferably identify a checksum for data integrity, along with potentially additional features including, but not limited to, calibration information, addressing capability, programming, and multiple parameters such as temperature, pressure, flow, pH, etc. Start and stop patterns are defined as well as the transmission rate and bit order for encoding, which will determine the signal to noise immunity vs. bandwidth tradeoff.

Using the IEC15693 standard for contactless vicinity ID cards as starting point, a communication protocol suitable for using in the monitoring system 50 may include the following features. The reader unit 80 initially requests the sensing device 10, 30 or 60 to respond, there is a start and end of frame for each communication direction, the digital data rate may be changed to ascertain distance, provisions for analog modulation are included to simplify implant electronics, and identification information is transmitted for responses from each sensing device (if the system 50 contains multiple sensing devices). FIG. 8 represents a suitable sequence, which begins with a start-of-frame (SOF) and is followed by parameter information that describes the data it precedes. The sequence finishes with an end-of-frame (EOF). The same basic sequence can be used for power and data transmission between reader unit and sensing device.

Communication from the reader unit 80 to the sensing device 10, 30 or 60 can be accomplished by suppressing the RF power from the reader unit 80 for short periods of time (reset). FIG. 9 represents an exemplary timing for this protocol. The reader unit 80 is the first to communicate, so that multiple sensing devices (if present) do not interfere with each other and corrupt the signal the reader unit 80 is attempting to read. A simplified version of the full protocol may include the following: only one 4-bit word (16 options) for parameters (a parameter selects which sensing device is to respond, no data transmission follows the parameters, the sensing device responds after the selection is made), no EOF, and all sensing devices respond unless asked not to.

As previously stated, the communication from the sensing device 10, 30 or 60 to the reader unit 80 can take place on a subharmonic carrier (3.39 MHz) of the power RF signal (13.56 MHz). The 3.39 MHz can be 100% amplitude modulated at various rates to determine the logic values and the framing. The protocol is preferably comprehensive, in that it allows for both digital and analog signal transmission and allows for future design flexibility in assigning codes, data types, and data bandwidth. As noted above, framing can be the same as discussed above in reference to FIG. 8 (SOF, Parameters, Data, EOF). A nonlimiting example of a suitable modulation for the digital portion of the transmission is as follows: data is 32 bits wide (parameters may include calibration, sensor identification, CRC (cyclic redundancy check), and/or data rate); logic 0 (nominal data rate)—48 cycles of 70.625 kHz (3.39 MHz/48); logic 1 (nominal data rate)—72 cycles of 105.9375 kHz (3.39 MHz/32), SOF—108 cycles of 105.9375 kHz followed directly by 72 cycles of 70.625 kHz followed directly by logic 1 followed directly by logic 0; and EOF—logic 0 followed directly by logic 1 followed directly by 72 cycles of 70.625 kHz followed directly by 108 cycles of 105.9375 kHz.

In addition to advantages associated with the transmission of both digital and analog data, such as improved accuracy and greater communication distance by allowing optimization of the antennas 64, 82 and 84, the wireless communication platform outlined above provides a comprehensive communication platform (including modulation scheme and modulation protocol) capable of addressing and communicating with a large number of different sensing devices 10, 30 or 60. In particular, the platform as described allows for communication with up to 256 sensing devices, with greater numbers achievable with appropriate modifications. In addition, the communication protocol can achieve the following: bi-directional communication, simultaneous and continuous tele-powering and tele-communication, high-speed communication (for example, greater than two hundred samples per second), greater insensitivity to the implant orientation in regards to the readout unit, ease of hardware implementation in an ASIC within the sensing device 10, 30 or 60, and minimal size of the sensing device 10, 30 or 60.

A wide variety of potential applications exist for the monitoring system, implantable sensing devices, and reader units of the types described above. Commercial applications include those in the medical field, and particularly applications that entail chronic or continuous measurements of physiological parameters, for example, in support of the trend toward home health monitoring. Particular examples include the diagnosis and/or monitoring of significant disease conditions, including congestive heart failure (CHF), hydrocephalus disease, and glaucoma disease. Other commercial applications encompass virtually any area that is in need of wireless sensing, for example, monitoring fluids in aerospace, automotive and industrial applications, including the monitoring of such physical and chemical parameters as pressure, flow, density, pH, and chemical composition of fluids, temperature, humidity, oxygen concentration, acceleration, radiation, etc. Military and governmental applications also exist that involve sensing of the above-noted physiological, physical and chemical parameters. As particular but nonlimiting examples, potential applications within the National Aeronautics and Space Administration (NASA) of the USA include implantable sensors for monitoring biological pressures in space and centrifuge-based systems, supporting animal studies of fundamental biological processes in cardiovascular, neurological, urological, and gastroenterological systems, monitoring effect of gravity or high accelerations on biological pressures, sensors requiring minimal power that can non-invasively measure pressure in environments with different gravity ranges, wireless sensors for remotely monitoring physical or chemical parameters in sealed containers, wireless telemetry communication for micro-biochemical and physical instruments and sensors, miniaturization of instruments through integration with MEMS-based sensors, in situ measurement and real time control of biological and physical phenomena, capability for automated acquisition, processing, and communication of biological data, miniature bio-processing systems that allow for precise measurement and closed loop control of multiple environmental parameters such as temperature, pH, oxygen, etc., and multiple intelligent implanted sensors that are addressable by a readout unit in a single or multiple animals in one or more environments.

While the invention has been described in terms of specific embodiments, it is apparent that other forms could be adopted by one skilled in the art. Therefore, the scope of the invention is to be limited only by the following claims.

1. A communication system for dynamically controlling power telemetrically delivered by a reader unit to a separate sensing device, the communication system comprising:
at least one telemetry antenna within the reader unit and adapted for electromagnetically delivering power to the sensing device;
at least one sensing element within the sensing device for sensing at least one parameter and producing an output based on the parameter;
at least one telemetry antenna within the sensing device for receiving the power electromagnetically delivered by the reader unit and for communicating signals from the sensing device to the reader unit; and
means for controlling the power supplied to the sensing device within a predetermined operating power level range of the sensing device.

2. The communication system according to claim 1, wherein the controlling means comprises means within the reader unit for evaluating a feedback signal at least partially derived from the signals of the sensing device and altering the power electromagnetically delivered by the reader unit to the sensing device.

3. The communication system according to claim 2, wherein the feedback signal is an internal receiver signal characteristic of a data signal of the sensing device.

4. The communication system according to claim 3, wherein the internal receiver signal evaluated by the evaluating means is chosen from the group consisting of receive signal strength indicator (RSSI), signal-to-noise ratio (S/N), signal-to-carrier ratio (S/C), a minimum or desired detectable signal strength, and combinations thereof.

5. The communication system according to claim 3, wherein the internal receiver signal evaluated by the evaluating means is a digital signal.

6. The communication system according to claim 3, wherein the internal receiver signal evaluated by the evaluating means is an analog signal.

7. The communication system according to claim 1, wherein the controlling means is located entirely within the reader unit.

8. The communication system according to claim 1, wherein the controlling means is located entirely within the sensing device.

9. The communication system according to claim 1, wherein the controlling means is located within both the sensing device and the reader unit.

10. The communication system according to claim 1, wherein the controlling means comprises a plurality of different controlling means.

11. The communication system according to claim 1, wherein the controlling means comprises means within the sensing device for generating an interactive signal and means within the reader unit for evaluating the interactive signal generated by the sensing device and altering the power electromagnetically delivered by the reader unit to the sensing device.

12. The communication system according to claim 11, wherein the interactive signal generated by the sensing device corresponds to a portion of the power electromagnetically delivered by the reader unit and received by the at least one telemetry antenna within the sensing device.

13. The communication system according to claim 12, wherein the generating means within the sensing device unit comprises means for assessing the quantity of the power received by the at least one telemetry antenna of the sensing device, and means for encoding information corresponding to the quantity on the signals communicated by the sensing device to the reader unit.

14. The communication system according to claim 1, wherein the controlling means comprises means within the sensing device for modifying the power electromagnetically delivered by the reader unit to the sensing device to a level within the operating power level range of the sensing device.

15. The communication system according to claim 14, wherein the modifying means comprises means within the sensing device for varying a tank load resistance and/or reactance of the at least one telemetry antenna of the sensing device.

16. The communication system according to claim 1, wherein the sensing device comprises means for combining digital and analog data to produce the signals of the sensing device.

17. The communication system according to claim 16, wherein the signals of the sensing device comprise a digital transmission characterized by digital modulation of an analog frequency.

18. The communication system according to claim 16, wherein the signals of the sensing device comprise an analog transmission characterized by analog modulation of an analog frequency.

19. The communication system according to claim 1, wherein the sensing device is adapted to sense a physiological parameter within a living body.

20. The communication system according to claim 19, wherein the physiological parameter is at least one pressure chosen from the group consisting of intraocular, intracranial, cardiovascular, and bariatric pressures.

21. The communication system according to claim 1, wherein the sensing device is adapted to sense at least one physical and/or chemical parameter in a medical. aerospace, automotive or industrial application.

22. The communication system according to claim 21, wherein the at least one physical and/or chemical parameter is at least one chosen from the group consisting of pressure, flow, density, pH, and chemical composition of a fluid, temperature, humidity, oxygen concentration, acceleration, and radiation.

23. The communication system according to claim 1, wherein the sensing device and reader unit are wirelessly coupled for telemetric communication using a passive scheme in which the sensing device receives power from the readout device only.

24. The communication system according to claim 1, wherein the sensing device contains a rechargeable power storage unit that receives power from and is recharged by the power electromagnetically delivered by the readout device to the sensing device.

25. The communication system according to claim 24, wherein the sensing device further contains a battery.

26. The communication system according to claim 1, wherein the sensing device contains electronic components for processing the output of the sensing element and generating therefrom the signals of the sensing device, the electronic components being adapted to be powered at an operating power level within the operating power level range of the sensing device, at least one of the electronic components being susceptible to heating if the at least one electronic component is supplied power that exceeds the operating power level, and the controlling means is adapted to prevent the power supplied to the electronic components from exceeding the operating power level of the at least one electronic component.

27. The communication system according to claim 1, wherein the communication system is installed in a medical system adapted to perform at least one of the following medical procedures: diagnosis, treatment intervention, tailoring of medications, disease management, identification of complications, and chronic disease management.

28. The communication system according to claim 1, wherein the reader unit is installed in a medical system adapted to perform at least one of the following: remote monitoring of a patient, closed-loop drug delivery of medications to treat a patient, warning of changes in the physiological parameter, portable or ambulatory monitoring or diagnosis, monitoring of battery operation, data storage, reporting global positioning coordinates for emergency applications, and communication with other medical devices.

29. A communication method for dynamically controlling power telemetrically delivered by a reader unit to a separate sensing device, the sensing device comprising at least one sensing element for sensing at least one parameter and producing an output based on the parameter, the sensing device generating signals from the output, and the method comprising:
electromagnetically delivering power from at least one telemetry antenna within the reader unit to at least one telemetry antenna within the sensing device; and
controlling the power supplied to the sensing device within a predetermined operating power level range of the sensing device.

30. The communication method according to claim 29, wherein the controlling step comprises evaluating a feedback signal at least partially derived from the signals of the sensing device and altering the power electromagnetically delivered by the reader unit to the sensing device.

31. The communication method according to claim 30, wherein the feedback signal is an internal receiver signal characteristic of a data signal of the sensing device.

32. The communication method according to claim 31, wherein the internal receiver signal is chosen from the group consisting of receive signal strength indicator (RSSI), signal-to-noise ratio (S/N), signal-to-carrier ratio (S/C), a minimum or desired detectable signal strength, and combinations thereof.

33. The communication method according to claim 31, wherein the internal receiver signal is a digital signal.

34. The communication method according to claim 31, wherein the internal receiver signal is an analog signal.

35. The communication method according to claim 29, wherein the controlling step is performed entirely within the reader unit.

36. The communication method according to claim 29, wherein the controlling is performed entirely within the sensing device.

37. The communication method according to claim 29, wherein the controlling is performed within both the sensing device and the reader unit.

38. The communication method according to claim 29, wherein the controlling step is performed by a plurality of different controlling means.

39. The communication method according to claim 29, wherein the controlling step comprises generating an interactive signal within the reader unit, evaluating the interactive signal within the reader unit, and altering the power electromagnetically delivered by the reader unit to the sensing device.

40. The communication method according to claim 39, wherein the interactive signal generated by the sensing device corresponds to a portion of the power electromagnetically delivered by the reader unit and received by the at least one telemetry antenna within the sensing device.

41. The communication method according to claim 40, wherein the generating step comprises assessing the quantity of the power received by the at least one telemetry antenna of the sensing device, and encoding information corresponding to the quantity on the signals communicated by the sensing device to the reader unit.

42. The communication method according to claim 29, wherein the controlling step comprises modifying within the sensing device the power electromagnetically delivered by the reader unit to the sensing device to a level within the operating power level range of the sensing device.

43. The communication method according to claim 42, wherein the modifying step comprises varying a tank load resistance and/or reactance of the at least one telemetry antenna of the sensing device.

44. The communication method according to claim 29, wherein the sensing device combines digital and analog data to produce the signals of the sensing device.

45. The communication method according to claim 44, wherein the signals of the sensing device comprise a digital transmission characterized by digital modulation of an analog frequency.

46. The communication method according to claim 29, wherein the signals of the sensing device comprise an analog transmission characterized by analog modulation of an analog frequency.

47. The communication method according to claim 46, wherein the sensing device is implanted within a living body and senses at least one physiological parameter within the living body.

48. The communication method according to claim 47, wherein the physiological parameter is at least one pressure chosen from the group consisting of intraocular, intracranial, cardiovascular and bariatric pressures.

49. The communication method according to claim 47, wherein the communication method is performed in at least one of the following medical procedures: diagnosis, treatment intervention, tailoring of medications, disease management, identification of complications, and chronic disease management.

50. The communication method according to claim 47, wherein the method further comprises using the reader unit to perform at least one of the following: remote monitoring of a patient, closed-loop drug delivery of medications to treat a patient, warning of changes in the physiological parameter, portable or ambulatory monitoring or diagnosis, monitoring of battery operation, data storage, reporting global positioning coordinates for emergency applications, and communication with other medical devices.

51. The communication method according to claim 29, wherein the sensing device senses at least one physical and/or chemical parameter of a fluid in a medical, aerospace, automotive or industrial application.

52. The communication method according to claim 51, wherein the at least one physical and/or chemical parameter is at least one chosen from the group consisting of pressure, flow, density, pH, and chemical composition of a fluid, temperature, humidity, oxygen concentration, acceleration, and radiation.

53. The communication method according to claim 29, wherein the sensing device and reader unit telemetrically communicate using a passive scheme in which the sensing device receives power from the readout device only.

54. The communication method according to claim 29, wherein the sensing device contains a rechargeable power storage unit that receives power from and is recharged by the power electromagnetically delivered by the readout device to the sensing device.

55. The communication method according to claim 29, wherein the sensing device contains a battery that receives power from and is recharged by the power electromagnetically delivered by the readout device to the sensing device.

56. The communication method according to claim 29, wherein the sensing device contains electronic components for processing the output of the sensing element and generating therefrom the signals of the sensing device, the electronic components being adapted to be powered at an operating power level within the operating power level range of the sensing device, at least one of the electronic components being susceptible to heating if the at least one electronic component is supplied power that exceeds the operating power level, and the controlling step comprises preventing the power supplied to the electronic components from exceeding the operating power level of the at least one electronic component.
